(19) United States
(12) Patent Application Publication
(10) Pub. No.: US 2016/0367182 A1
(43) Pub. Date: Dec. 22, 2016

(54) PERSPIRATION MONITORING

(71) Applicant: EMPIRE TECHNOLOGY DEVELOPMENT LLC, Wilmington, DE (US)

(72) Inventors: Benjamin William Millar, Rosebery (AU); George Charles Peppou, Rosebery (AU)

(21) Appl. No.: 14/741,328

(22) Filed: Jun. 16, 2015

(51) Int. Cl. A61B 5/00

(52) U.S. Cl. CPC A61B 5/4266; A61B 5/6801; A61B 5/7405; A61B 5/742; A61B 5/7455

(57) ABSTRACT

A system may include a mesh that includes a non-electrically conductive mesh material. The system may also include a first electrode coupled to the mesh at a first location of the mesh. The system may further include a second electrode coupled to the mesh at a second location of the mesh that is different from the first location. Additionally, the system may include a control unit electrically coupled to the first electrode and the second electrode. The control unit may be configured to generate a notification signal when the first electrode and the second electrode are electrically coupled to each other.

### BACKGROUND

Unless otherwise indicated, the materials described herein are not prior art to the claims in the present application and are not admitted to be prior art by inclusion in this section.

Participants of physical activity often perspire and thus lose fluids and electrolytes during and after the physical activity. Consuming liquids such as water or sports drinks may help replenish the lost fluids and electrolytes. However, participants are often unaware of the amount of fluids lost during physical activity or of how often to hydrate.

### SUMMARY

Technologies described herein generally relate to perspiration monitoring.

In some examples, a method is described. The method may include detecting whether perspiration disposed along a mesh electrically couples a first electrode and a second electrode that are coupled to the mesh at different locations of the mesh. The method may also include generating a notification signal when the perspiration electrically couples the first electrode and the second electrode.

In some examples, a system is described. The system may include a mesh that includes a non-electrically conductive mesh-material. The system may also include a first electrode coupled to the mesh at a first location of the mesh. The system may further include a second electrode coupled to the mesh at a second location of the mesh that is different from the first location. Additionally, the system may include a control unit electrically coupled to the first electrode and the second electrode. The control unit may be configured to generate a notification signal when the first electrode and the second electrode are electrically coupled to each other.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings. In the drawings:

FIG. 1 illustrates an example system configured to detect a perspiration rate and generate a notification based on the perspiration rate;

FIG. 2 illustrates another example system configured to detect a perspiration rate and generate a notification based on the perspiration rate;

FIG. 3A illustrates another example system configured to detect a perspiration rate and generate a notification based on the perspiration rate;

FIG. 3B illustrates another view of the example system of FIG. 3A;

FIG. 4 illustrates a flow diagram of an example method of detecting perspiration and generating a corresponding hydration notification; and

FIG. 5 is a block diagram illustrating an example computing device that is arranged to direct one or more operations of detecting perspiration and generating a corresponding hydration notification; all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, and devices that relate to monitoring an amount of perspiration of a subject. Further, based on the monitored perspiration, a notification may be generated that indicates a recommendation for the subject to hydrate.

In some embodiments, a device may detect a rate of perspiration of the subject and may generate a notification signal when the rate of perspiration is above that of a target threshold. The notification signal may be received by a notification unit that may generate a notification that may indicate a recommendation to the subject to hydrate. Therefore, the subject may be reminded to hydrate to help replenish fluids and/or electrolytes that may be lost through the perspiration. The reminder may help reduce the likelihood of dehydration, cramping, fatigue, or any other condition that may result from not having enough fluids in the body.

Reference is now made to the drawings.

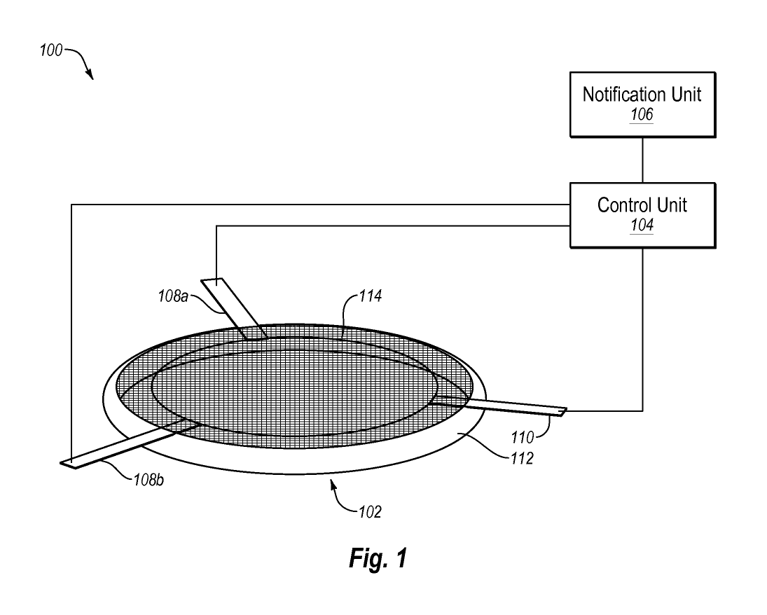

FIG. 1 illustrates a system 100 configured to detect a perspiration rate and generate a notification based on the perspiration rate, arranged in accordance with at least some embodiments described herein. In the illustrated embodiment, the system 100 may include a detection device 102, a control unit 104, and a notification unit 106.

The detection device 102 may include one or more first electrodes and one or more second electrodes. In the illustrated example, the detection device 102 may include a first electrode 108a, a first electrode 108b, and a second electrode 110. The detection device 102 may also include a frame 112 and a mesh 114.

The frame 112 may include any suitable apparatus that may outline and define a space. Additionally, the frame 112 may include any non-electrically conductive material such as silicone, rubber, nylon, plastic, other natural or synthetic polymers, etc.

For example, in some embodiments, the frame 112 may include a silicone ring such as depicted in FIG. 1. Although the frame 112 is depicted with a circular shape, the frame 112 may include any suitable shape. For example, the frame 112 may include a rectangular shape, an oval shape, a trapezoidal shape, an octagonal shape, a pentagonal shape, a hexagonal shape, or any other suitable shape.

In some embodiments, the mesh 114 may be mechanically coupled to the frame 112 such that the mesh 114 may extend across the space that may be defined by the frame 112. Additionally, the mesh 114 may be mechanically coupled to the frame 112 such that when the frame 112 is placed against a surface (e.g., a skin surface of a subject) a void may be created and enclosed between the mesh 114 and the surface. The void may also be enclosed by the frame 112 and the distance between the mesh 114 and the surface may be based on a thickness of the frame 112. In the illustrated example, the mesh 114 is depicted as being coupled to a side of the frame 112 such that the mesh 114 substantially covers a side of the space outlined by the frame 112. Additionally or alternatively, the mesh 114 may be coupled to an interior portion of the frame 112 such that the mesh 114 may extend across and within the space that may be outlined by the frame 112. A side of the mesh 114 that faces the void may be referred to as the inner side of the mesh 114 and a side of the mesh 114 opposite the inner side of the mesh 114 may be referred to as the outer side of the mesh 114.

In some examples, the frame 112 may include a deformable portion configured to contact the surface, and a relatively more rigid portion configured to support the mesh 114. In some embodiments, the frame 112 may be configured to attach or be to the surface. For example, the frame 112 may include an adhesive layer, allowing the frame to be adhered to the surface. In some examples, the surface may be a vapor-exuding surface, such as a portion of the skin of a living subject that exudes sweat (such as a portion of a human skin, or a dog's paw pad).

The mesh 114 may include a mesh material that is non-electrically conductive and that is hydrophobic. The hydrophobic nature of the mesh material may repel droplets of a liquid, which may prevent or reduce absorption of the liquid (e.g., water or perspiration) by the mesh 114. Therefore, in some embodiments, the mesh 114 may be configured such that a liquid may be disposed along a surface of the inner side of the mesh when the void includes a particular amount of liquid while the mesh 114 may also not substantially absorb the liquid. The mesh 114 may include a molded film, a non-woven or woven fabric, or in some examples a material such as a thin film having holes disposed therethrough.

When the frame 112 is placed against the skin surface of a subject who is perspiring, the moisture and perspiration within the void between the skin surface and the inner side of the mesh 114 may increase. In some instances, the amount of perspiration may be such that the perspiration may be disposed along a substantial portion of the inner side of the mesh 114 by filling the void. As described in further detail below, the system 100 may generate a notification that indicates a recommendation to hydrate based on the amount of perspiration disposed along the inner side of the mesh 114.

The mesh 114 may also include a mesh pattern that may include multiple holes. The holes may allow for evaporation of moisture (e.g., perspiration) that may be disposed within the void between the skin surface and the mesh 114. In some examples, the holes may be sized such that a surface tension across them may restrict the flow of liquid from inside the void to outside of the void through the holes without external force (provided by movement) or evaporation. In some embodiments, the size of the holes of the mesh 114 may thus be based on a target evaporation rate of moisture that may be disposed within the void between the mesh 114 and the skin surface. In some embodiments, the target evaporation rate may be based on a target perspiration rate associated with generating the notification to hydrate. In some embodiments, the holes may have a hole diameter, for example in the range 0.01 mm-2 mm.

For example, as indicated above and described in further detail below, in some embodiments, the system 100 may be configured to generate the notification signal based on the amount of perspiration disposed along the inner side of the mesh 114, which may be a function of the amount of perspiration within the void. The amount of perspiration within the void, and thus disposed along the inner side of the mesh 114, may be based on the perspiration rate of the subject. However, the amount of perspiration disposed along the inner side of the mesh 114 may also be based on the evaporation rate and the ejection rate of the perspiration within the void between the mesh 114 and the skin surface.

Therefore, if the perspiration rate is relatively low as compared to the evaporation rate, the amount of perspiration disposed along the inner side of the mesh 114 may be relatively low such that, in some embodiments and instances, the system 100 may not generate the notification signal. Conversely, if the perspiration rate is relatively high as compared to the evaporation rate, the amount of perspiration disposed along the inner side of the mesh 114 may be relatively high such that, in some embodiments and instances, the system 100 may generate the notification signal. Therefore, in some embodiments, the size of the holes in the mesh 114 may be configured based on a target perspiration rate for generating the notification signal.

In some instances, the amount of distance between the skin surface and the mesh 114 may also affect the amount of perspiration that may be disposed along the inner side of the mesh 114. For example, the amount of distance between the skin surface and the mesh 114 may affect the amount of time that it takes for perspiration to fill the void and reach the mesh 114. Additionally, this amount of time may vary according to the perspiration rate. Therefore, in some embodiments, the mesh 114 and the frame 112 may be configured such that the distance between the mesh 114 and the skin surface is such that perspiration may reach the mesh 114 if the perspiration rate is greater than the rate of loss through the mesh, as the void may then be filled. The mesh loss rate may thus be tuned to determine a specific perspiration rate threshold associated with generating the notification. The amount of distance between the skin surface and the mesh 114 may be based on placement of the mesh 114 with respect to the frame 112 and/or based on the thickness of the frame 112.

The first electrodes 108a and 108b may be coupled to the mesh 114 at differing locations, such as illustrated in FIG. 1. In some embodiments, such as illustrated in FIG. 1, the first electrodes 108a and 108b may be coupled to the mesh 114 on a side of the mesh 114 that faces a surface when the frame 112 is disposed against the surface (e.g., the inner side of the mesh 114). The first electrodes 108a and 108b may include any suitable material that may be electrically conductive.

The second electrode 110 may also be coupled to the mesh 114 at a location that is different from the locations of the first electrode 108a and the first electrode 108b.

In some embodiments, the second electrode 110 may be coupled to the mesh 114 on the side of the mesh 114 that faces the surface when the frame 112 is disposed against the surface (e.g., the inner side of the mesh 114), similar to the first electrodes 108a and 108b.

In some instances, when at least a certain amount of liquid substance (e.g., perspiration) is disposed along the inner side of the mesh 114 between one or more of the first electrodes 108 and the second electrode 110, the liquid substance may electrically couple one or more of the first electrodes 108 with the second electrode 110. When the frame 112 is against the skin surface of the subject, a number of first electrodes 108 that are electrically coupled to the second electrode 110 may indicate a perspiration rate of the subject.

For example, a relatively large amount of perspiration disposed along the inner side of the mesh 114 may electrically couple both the first electrode 108a and the first electrode 108b with the second electrode 110. By comparison, a smaller amount of perspiration disposed along the inner side of the mesh 114 may electrically couple the first electrode 108a with the second electrode 110 but may not be of a sufficient amount to also electrically couple the first electrode 108b with the second electrode 110, or vice versa. Additionally, an even smaller amount of perspiration (e.g., little to no perspiration) disposed along the inner side of the mesh 114 may be such that neither the first electrode 108a nor the first electrode 108b is electrically coupled to the second electrode 110. Therefore, the number of first electrodes 108 electrically coupled to the second electrode 110 may indicate the amount of perspiration disposed along the inner side of the mesh 114, which may indicate the amount of perspiration in the corresponding void between the skin surface and the mesh, which in turn indicate the perspiration rate of the subject.

A degree of electrical coupling between one or more of the first electrodes 108 and the second electrode 110 may also indicate the perspiration rate of the subject. For example, a relatively large amount of perspiration between the first electrode 108a and the second electrode 110 may provide a greater amount of electrical coupling (e.g., less electrical resistance) between the first electrode 108a and the second electrode 110 than if less perspiration were between the first electrode 108a and the second electrode 110. The same principle may apply with respect to the amount of perspiration between the first electrode 108b and the second electrode 110. Therefore, the degree of electrical coupling between the first electrodes 108 and the second electrode 110 may indicate the amount of perspiration disposed along the inner side of the mesh 114, which may in turn indicate the perspiration rate of the subject.

Additionally, in some embodiments, the spacing between the first electrodes 108 and the second electrode 110 and whether or not they are electrically coupled by perspiration may indicate the perspiration rate of the subject. For example, when the first electrodes 108 are relatively close to the second electrode 110, less perspiration may be present to electrically couple the first electrodes 108 to the second electrode than when the first electrodes 108 are relatively far away from the second electrode 110. Therefore, in some embodiments, the placement of the first electrodes 108 with respect to the second electrode 110 may be selected based on a target perspiration rate that may be determined. Additionally, in some embodiments, the spacing between the first electrode 108a and the first electrode 108b with respect to the second electrode 110 may differ. Therefore, in some embodiments, electrical coupling between the first electrode 108a and the second electrode 110 may indicate a different perspiration rate than electrical coupling between the first electrode 108b and the second electrode 110 and electrical coupling between both first electrodes 108 and the second electrode 110 may indicate an even different perspiration rate.

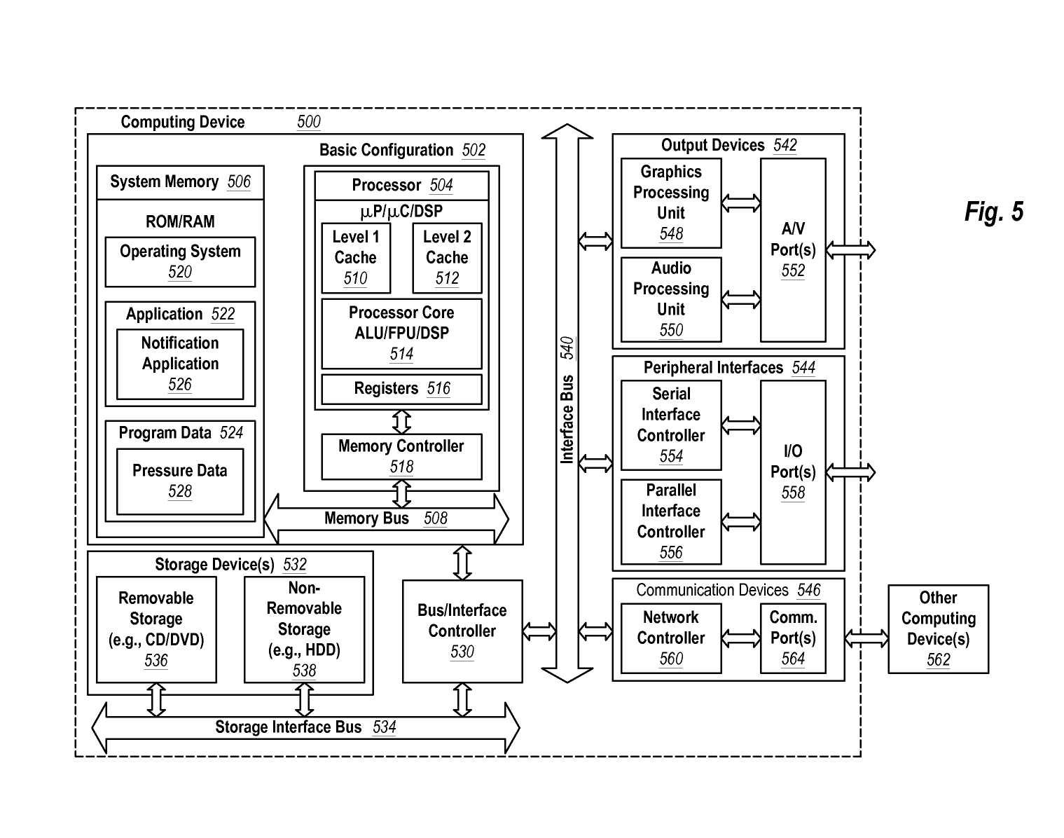

The control unit 104 may include any suitable system, apparatus, or device that may be configured to perform operations associated with generating a notification signal based on electrical coupling between one or more of the first electrodes 108 and the second electrode 110 that may be from a liquid substance (e.g., perspiration) disposed along the inner side of the mesh 114. A computing device 500 described below with respect to FIG. 5 is an example embodiment of the control unit 104.

In some embodiments, the control unit 104 may be configured to determine whether one, some, or all of the first electrodes 108 are electrically coupled to the second electrode 110. Additionally or alternatively, the control unit 104 may be configured to determine a number of first electrodes 108 that are electrically coupled to the second electrode 110. In these or other embodiments, the control unit 104 may be configured to determine a degree of electrical coupling between one or more of the first electrodes 108 and the second electrode 110. As indicated above, these determinations may indicate a perspiration rate of the subject. Therefore, in some embodiments, the control unit 104 may be configured to generate the notification signal based on one or more of the above-mentioned determinations.

In some embodiments, the control unit 104 may be configured to determine resistance between each of the first electrodes 108 and the second electrode 110. The determined resistance may indicate which of the first electrodes 108 may be electrically coupled to the second electrode 110 and/or a degree of electrical coupling between the first electrodes 108 and the second electrode 110. In particular, the greater the amount of resistance between the first electrodes 108 and the second electrode 110, the less the amount of electrical coupling. Therefore a relatively large amount of resistance may indicate little to no electrical coupling and a relatively small amount of resistance may indicate electrical coupling. As indicated above, whether or not the first electrodes 108 are electrically coupled to the second electrode 110 and/or the degree of electrical coupling may indicate an amount of perspiration disposed along the inner side of the mesh 114. Therefore, in some instances, the resistance that may be measured by the control unit 104 may indicate a perspiration rate of a subject.

In some embodiments, the control unit 104 may be configured to generate the notification signal based on the determination of electrical coupling between the first electrodes 108 and the second electrode 110, which may indicate the perspiration rate of the subject. As described below and indicated above, the notification signal may be communicated to the notification unit 106 such that the notification unit 106 may generate a notification that indicates a recommendation to hydrate. As the perspiration rate of the subject increases, the amount and/or frequency of recommended hydration may also increase. Therefore, in some embodiments, the control unit 104 may be configured to vary or generate the notification signal based on the determination of electrical coupling between the first electrodes 108 and the second electrode 110.

For example, in some embodiments, the control unit 104 may be configured to generate the notification signal when both the first electrode 108a and the first electrode 108b are determined to be electrically coupled to the second electrode 110, which may indicate that the perspiration rate is at or above a certain level. In these or other embodiments, the control unit 104 may be configured to periodically generate the notification signal over a first time interval over which both the first electrode 108a and the first electrode 108b are electrically coupled to the second electrode 110. The first time interval may be based on a first perspiration rate that may be estimated based on the electrical coupling of both the first electrode 108a and the first electrode 108b with the second electrode 110.

Additionally or alternatively, the control unit 104 may be configured to periodically generate the notification signal over a second time interval when one but not both of the first electrodes 108 are electrically coupled to the second electrode. The second time interval may be based on a second perspiration rate that may be estimated based on the electrical coupling of one but not both of the first electrodes 108 with the second electrode 110.

Additionally or alternatively, the control unit 104 may be configured to generate the notification signal one or more times after one or more of the first electrodes 108 are no longer electrically coupled to the second electrode 110. Therefore, the subject may be reminded to continue hydrating even after finishing an activity that may have caused perspiring or after reducing intensity of the activity. In these or other embodiments, the frequency of the notification signal after one or more of the first electrodes 108 are no longer electrically coupled to the second electrode 110 may be based on the number of first electrodes 108 that are still electrically coupled to the second electrode. In these or other embodiments, the frequency of the notification signal after one or more of the first electrodes 108 are no longer electrically coupled to the second electrode 110 may be based on a time interval that may be associated with a recommended hydration rate that may correspond with the perspiration rate that may be associated with the electrical coupling of the first electrodes 108.

As mentioned above, the degree of electrical coupling between the first electrodes 108 and the second electrode 110 from perspiration may also indicate the perspiration rate of a subject. Therefore, in some embodiments, the control unit 104 may be configured to generate the notification signal based on the degree of electrical coupling between the first electrodes 108 and the second electrode 110 and not just based on whether or not the first electrodes 108 are electrically coupled to the second electrode 110. As such, the control unit 104 may be configured to generate the notification signal based on different perspiration rates that may be associated with different degrees of electrical coupling.

By way of example, when the amount of resistance between a particular first electrode 108 and the second electrode 110 is at or above a threshold amount, the control unit 104 may be configured to determine that the particular first electrode 108 and the second electrode 110 are not electrically coupled by any appreciable degree. Accordingly, in some embodiments, the control unit 104 may not generate a notification signal associated with the particular first electrode 108 being electrically coupled to the second electrode 110.

Additionally or alternatively, when the amount of resistance between the particular first electrode 108 and the second electrode 110 is within a first resistance range determined to be associated with a first perspiration rate, the control unit 104 may generate the control signal based on the first perspiration rate. Moreover, when the amount of resistance between the particular first electrode 108 and the second electrode 110 is within a second resistance range determined to be associated with a second perspiration rate, the control unit 104 may generate the control signal based on the second perspiration rate. The number of resistance ranges, associated perspiration rates, and consequent variations on the control signal may vary.

In some embodiments, a determination of whether or not the particular first electrode 108 is electrically coupled to the second electrode 110 may provide a rough estimation of perspiration rate and a determination of actual resistance may provide a fine estimation of perspiration rate. Moreover, as the number of first electrodes 108 increases, the granularity of estimation of the perspiration rate based on a determination of the number of first electrodes 108 that may be electrically coupled to the second electrode 110 may also increase. In these or other embodiments, the increased granularity from increasing the number of first electrodes 108 may also be increased by determining amounts of resistance. Therefore, the notification signal may be varied according to different levels of granularity of estimations of perspiration rates that may be based on one or more of whether or not the particular first electrode 108 is electrically coupled to the second electrode 110, the number of first electrodes 108 electrically coupled to the second electrode 110, and an amount of resistance between one or more of the first electrodes 108 and the second electrode 110.

For example, both the first electrode 108a and the first electrode 108b being electrically coupled to the second electrode 110 may indicate that a perspiration rate is at or above a first level. Additionally, the first electrode 108a being electrically coupled to the second electrode 110 and the first electrode 108b not being electrically coupled to the second electrode 110, or vice versa may indicate that the perspiration rate is at or above a second level but below the first level. In this example, the resistance between the first electrode 108a and the second electrode 110 may vary between the second level and the first level of perspiration rates depending on the actual perspiration rate that may be between the second level and the first level. Therefore, the resistance measurement may indicate that the perspiration rate is at a third level that may be between the first level and the second level, thus giving more granularity in determining the perspiration rate.

In some instances a liquid substance other than perspiration may electrically couple one or more of the first electrodes 108 with the second electrode 110. For example, in some instances, rainwater may electrically couple the first electrodes 108 with the second electrode 110. However, rainwater may have a different chemical composition than perspiration such that the electrical conductivity of rainwater may be different from that of perspiration. Therefore, in some embodiments, the control unit 104 may be configured to generate the notification signal based on resistances that correspond to an electrical conductivity range associated with perspiration but not rainwater.

The notification unit 106 may be communicatively coupled to the control unit 104 such that the notification unit 106 may receive the notification signal generated by the control unit 104. The notification unit 106 may be communicatively coupled to the control unit 104 via any suitable wired or wireless coupling.

The notification unit 106 may include any suitable system, apparatus, or device configured to generate a notification in response to receiving the notification signal. For example, the notification unit 106 may include one or more of a speaker, a vibrator, and a light. In some embodiments, the notification unit 106 may include a portable electronic device such as a smartphone. The notification that may be generated by the notification unit 106 may include any suitable audible, visual, haptic, or any other cue that may indicate a recommendation to hydrate and that may be perceived by the subject. For example, the notification may include a sound, a vibration, a light indication, or any combination thereof.

Additionally, the notification may be simple or complex. For example, the notification may include a simple sound such as a beep or may be more complex in which a voice indicates a recommendation to hydrate. Additionally or alternatively, the notification may include a message on a display of an electronic device that may indicate the recommendation to hydrate and/or may include a blinking light. In these or other embodiments, the notification may include a vibration and/or a series of vibrations. Further, in some embodiments, the notification may include a message indicating a recommended amount of fluid intake and/or a recommended interval for fluid intake.

As indicated above, in some embodiments, the notification signal may vary based on different perspiration rates such that the notification may also vary based on different perspiration rates. For example, in some embodiments, the control unit 104 may increase or decrease the frequency of generating the notification signal based on perspiration rates (as indicated by the electrical coupling of the first electrodes 108 with the second electrode 110 described above) such that the frequency of the generation of the notification may vary based on perspiration rates. Additionally or alternatively, the notification signal may vary based on the perspiration rates such that the notification itself may vary. For example, the control unit 104 may generate the notification signal such that the notification unit 106 indicates a first recommended amount of fluid intake based on a first perspiration rate and may also generate the notification signal such that the notification unit 106 indicates a second recommended amount of fluid intake based on a second perspiration rate.

In some embodiments, the control unit 104 may be communicatively coupled to an electronic device (e.g. a smartphone with an accompanying app, a personal computer with an accompanying program, a tablet computer with an accompanying app etc.). The electronic device may or may not also be configured as the notification unit 106. In some embodiments, the control unit 104 may be configured to update perspiration rate information in real time to the electronic device. Additionally or alternatively, the control unit 104 may be configure to store perspiration rate information and to upload, when a connection with the electronic device is present; time-stamped data detailing time spent at or above the perspiration rate thresholds. This may be used to track exercise intensity and duration, compare with fluid intake, and aid in informed hydration management.

In some embodiments, the system 100 may include may include a strap, or other mechanical fastener (not expressly illustrated), configured to retain the detection device 102 in contact with the surface. For example, in some embodiments, the detection device 102 may be positioned similar to a watch, with the frame 112 sealed against the surface of the skin on a forearm by a secured band. The detection device 102 may be similarly mounted onto a subject's skin on the upper arm, chest, upper back, lower back, or leg, using bands sized according to their target placement area.

In these or other embodiments, the band may be attached to the rigid or partially rigid frame 112, extending from opposing lateral sides. Additionally or alternatively, the frame 112 may also be included in the band, such that the void may exist as a hole through a single strap of band material of certain depth, with the mesh 114 and the electrodes arranged over the hole. In these or other embodiments, other electronics (e.g., the control unit 104) may be mounted in or on the band. In some examples, the detection device 102 and/or the control unit 104 may be a component of, or attached to, another device with other functionality, such as a watch, a heart-rate monitor, a portable electronic device and the like.

In some embodiments, the system 100 may include a dedicated power source such as a battery. Additionally or alternatively, the system 100 may include a source of power such as a solar cell, electrolytic source of voltage (e.g. dissimilar metals in contact with a skin surface), and the like. In some examples, the system 100 may include a power source that converts an external electromagnetic radiation into usable power (e.g., a combination of coil, rectifier, capacitor, and/or other components that may provide a direct current power source, e.g., in response to a transponder, ambient electromagnetic fields, deformation of the skin (e.g. piezoelectric elements), and the like).

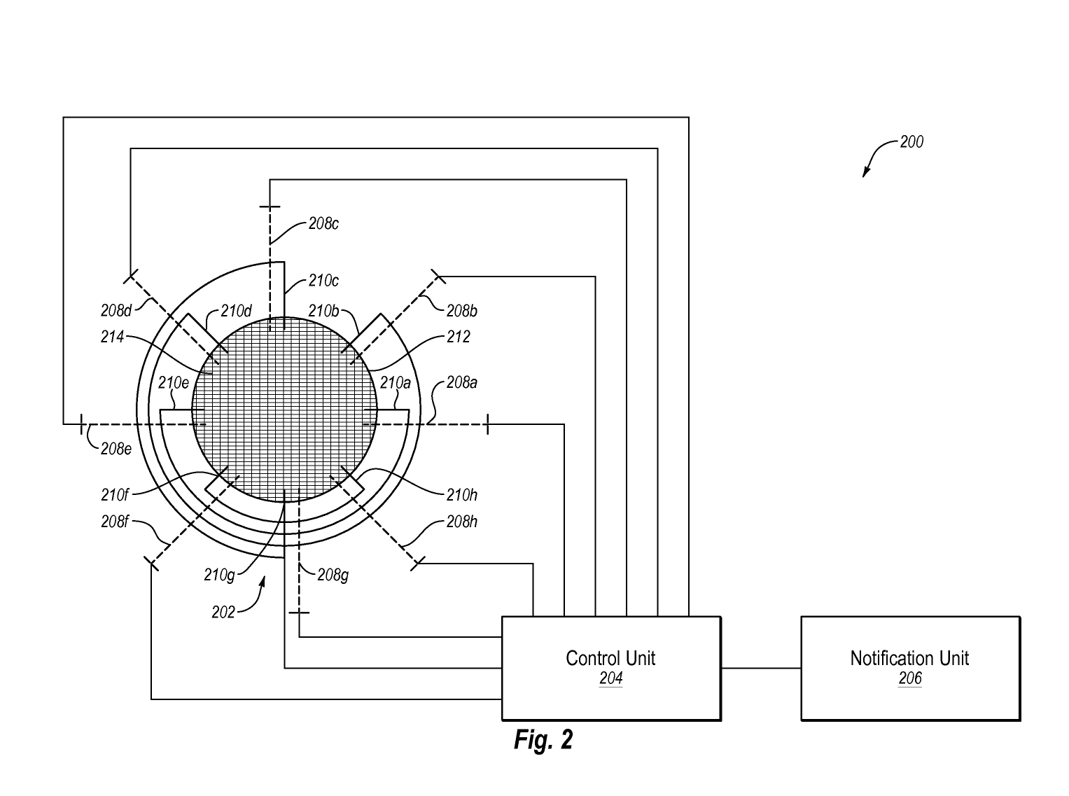

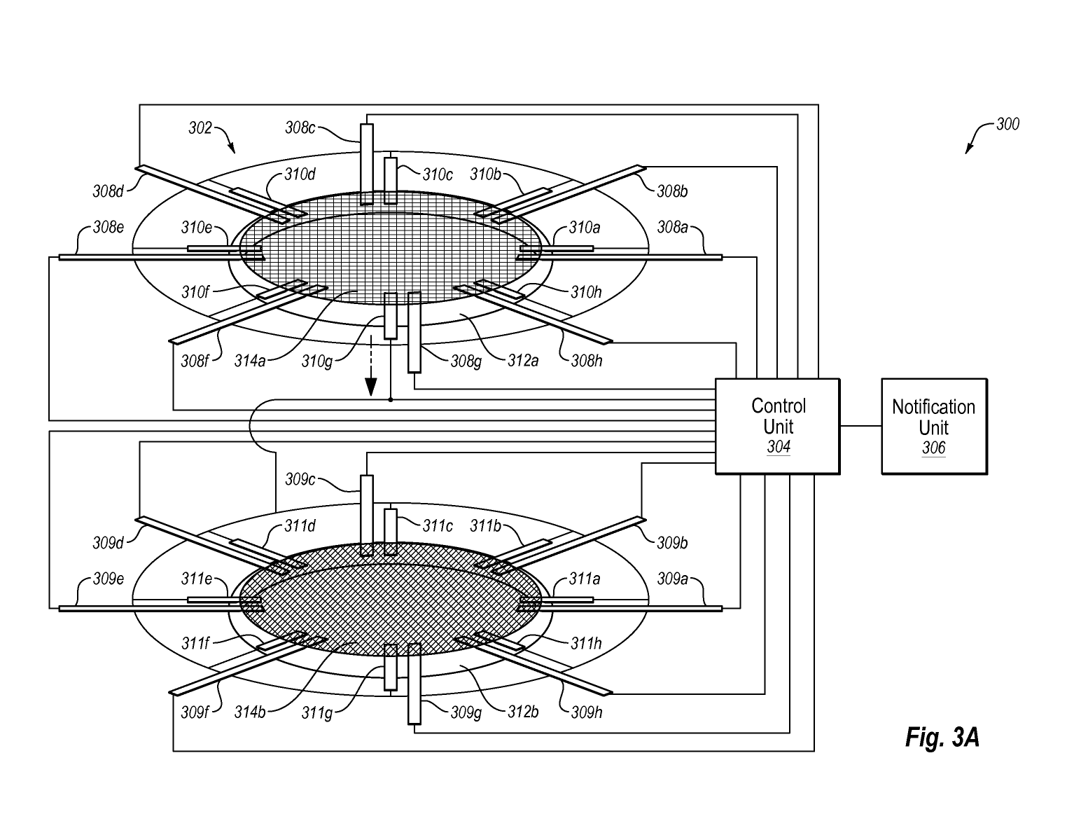

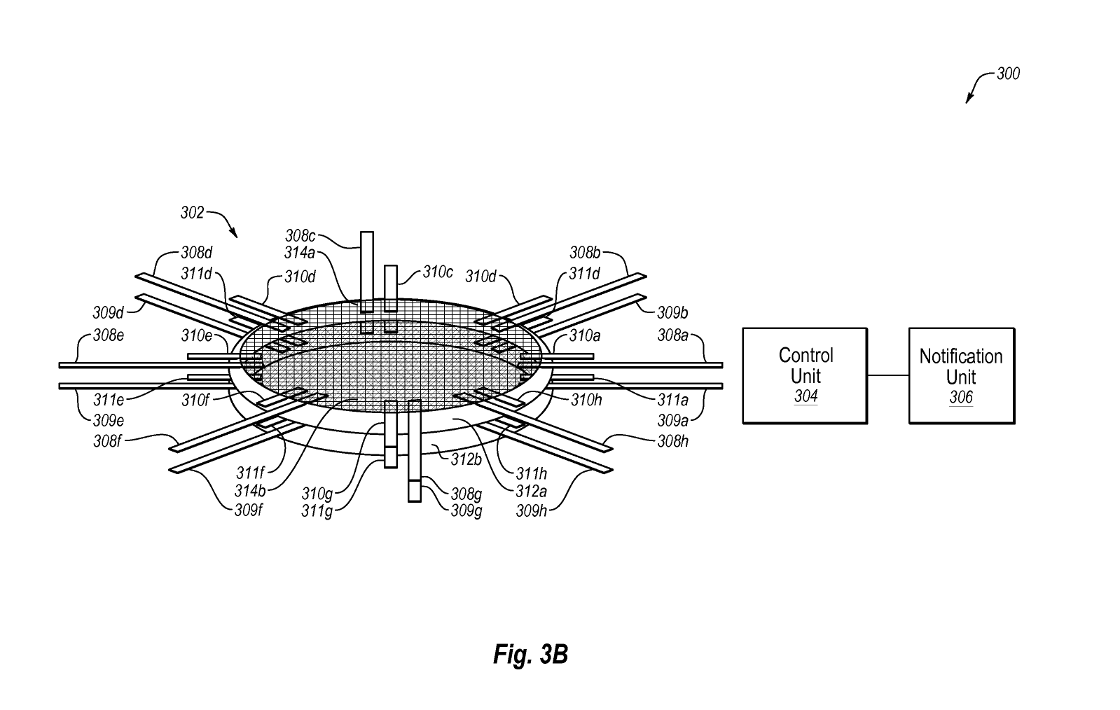

Modifications, additions, or omissions may be made to FIG. 1 without departing from the scope of the present disclosure. For example, the system 100 may include any number of other components not expressly illustrated or described. Further, the number of first electrodes and/or second electrodes may vary. For example, in some embodiments, a detection device may include more or fewer than two first electrodes and/or more than one second electrode. FIG. 2, which is described below, illustrates a detection device 202 with more first and second electrodes than the detection device 102 of FIG. 1. Additionally, in some embodiments, the detection device may include more than one mesh layer. FIGS. 3A and 3B, which are described below, illustrate a detection device 302 with an additional mesh layer as compared to the detection device 102.

FIG. 2 illustrates a system 200 configured to detect a perspiration rate and generate a notification based on the perspiration rate, arranged in accordance with at least some embodiments described herein. In the illustrated embodiment, the system 200 may include the detection device 202 mentioned above, a control unit 204, and a notification unit 206.

The detection device 202 may include a frame 212 and a mesh 214. The frame 212 may be analogous to the frame 112 of the detection device 102 of FIG. 1. Additionally, the mesh 214 may be analogous to the mesh 114 of the detection device 102 of FIG. 1.

The detection device 202 may also include one or more first electrodes and one or more second electrodes. In the illustrated example, the detection device 202 may include a first electrode 208a, a first electrode 208b, a first electrode 208c, a first electrode 208d, a first electrode 208e, a first electrode 208f, a first electrode 208g, and a first electrode 208h. Additionally, in the illustrated example, the detection device 202 may include a second electrode 210a, a second electrode 210b, a second electrode 210c, a second electrode 210d, a second electrode 210e, a second electrode 210f, a second electrode 210g, and a second electrode 210h.

The first electrodes 208 may be coupled to the mesh 214 at differing locations, such as illustrated in FIG. 2. Additionally, in the illustrated example, each of the second electrodes 210 may be coupled to the mesh 214 at a location that is proximate to one of the first electrodes 208 (e.g., as illustrated in FIG. 2) such that each first electrode 208 may correspond to the second electrodes 210. Further, the second electrodes 210 may be electrically coupled to each other such that they each may be associated with a same electrical node of the detection device 202.

For similar reasons as described above with respect to the detection device 102, a number of first electrodes 208 electrically coupled to one or more of the second electrodes 210 may indicate a perspiration rate of the subject. Additionally, a degree of electrical coupling between the first electrodes 208 and the second electrodes 210 may also indicate the perspiration rate of the subject for similar reasons described above with respect to the detection device 102.

The control unit 204 may be substantially analogous to the control unit 104 of FIG. 1. Therefore, the control unit 204 may be configured to determine the number of first electrodes 208 electrically coupled to one or more of the second electrodes 210 and may generate a notification signal according to perspiration rates that may correspond to the number of first electrodes 208 electrically coupled to one or more of the second electrodes 210, similar to as described above with respect to the control unit 104 generating the notification signal. Additionally or alternatively, the control unit 204 may be configured to determine a degree of electrical coupling between one or more of the first electrodes 208 and one or more of the second electrodes 210 (e.g., via resistance measurements) and may generate the notification signal according to perspiration rates that may correspond to the degree of coupling, similar to as described above with respect to the control unit 104 generating the notification signal.

The notification unit 206 may be substantially analogous to the notification unit 106 of FIG. 1. Therefore, the notification unit 206 may be configured to receive the notification signal generated by the control unit 204 and to generate a notification based on the notification signal. The notification may include one or more of the example notifications described above with respect to FIG. 1.

Modifications, additions, or omissions may be made to FIG. 2 without departing from the scope of the present disclosure. For example, the system 200 may include any number of other components not expressly illustrated or described. Further, the number of first electrodes and/or second electrodes may vary.

FIGS. 3A and 3B illustrate a system 300 configured to detect a perspiration rate and generate a notification based on the perspiration rate, arranged in accordance with at least some embodiments described herein. In the illustrated embodiment of FIGS. 3A and 3B, the system 300 may include the detection device 302 mentioned above, a control unit 304 and a notification unit 306. FIG. 3A illustrates an exploded view of the detection device 302 and FIG. 3B illustrates the detection device 302 in a coupled form.

The detection device 302 may include a first frame 312a, a second frame 312b, a first mesh 314a, and a second mesh 314b. The frames 312 may be analogous to the frame 112 of the detection device 102 of FIG. 1. Additionally, the meshes 314 may be analogous to the mesh 114 of the detection device 102 of FIG. 1.

In the illustrated example, the first mesh 314a may be coupled to the first frame 312a such that the first mesh 314a may extend across a space that may be defined by the first frame 312a. Similarly, the second mesh 314b may be coupled to the second frame 312b such that the second mesh 314b may extend across a space that may be defined by the second frame 312b. In some embodiments, the first frame 312a may be coupled to the second frame 312b in a configuration in which a first void may be created and enclosed between the first mesh 314a and the second mesh 314b. The first void may also be enclosed by the first frame 312a and a distance between the first mesh 314a and the second mesh 314b may be based on a thickness of the first frame 312a. In the present disclosure, a side of the first mesh 314a that may face the first void may be referred to as an inner side of the first mesh 314a.

Additionally, the second mesh 314b may be coupled to the second frame 312b such that when the second frame 312b is placed against a surface (e.g., a skin surface of a subject), a second void may be created and enclosed between the second mesh 314b and the surface. The second void may also be enclosed by the second frame 312b and the distance between the second mesh 314b and the surface may be based on a thickness of the second frame 312b. In the present disclosure, a side of the second mesh 314b that may face the second void may be referred to as an inner side of the second mesh 314b.

In the illustrated example, the first mesh 314a is depicted as being coupled to a side of the first frame 312a such that the first mesh 314a substantially covers a side of a first space outlined by the first frame 312a. Similarly, the second mesh 314b is depicted as being coupled to a side of the second frame 312b such that the second mesh 314b substantially covers a side of a second space outlined by the second frame 312b.

Additionally or alternatively, the first mesh 314a may be coupled to an interior portion of the first frame 312a such that the first mesh 314a may extend across and within the space that may be outlined by the first frame 312a. Similarly, the second mesh 314b may be coupled to an interior portion of the second frame 312b such that the second mesh 314b may extend across and within the space that may be outlined by the second frame 312b.

The detection device 302 may include one or more first electrodes and one or more second electrodes. In the illustrated example, the detection device 302 may include a first electrode 308a, a first electrode 308b, a first electrode 308c, a first electrode 308d, a first electrode 308e, a first electrode 308f, a first electrode 308g, and a first electrode 308h. Additionally, in the illustrated example, the detection device 302 may include a first electrode 309a, a first electrode 309b, a first electrode 309c, a first electrode 309d, a first electrode 309e, a first electrode 309f, a first electrode 309g, and a first electrode 309h. Further, in the illustrated example, the detection device 302 may include a second electrode 310a, a second electrode 310b, a second electrode 310c, a second electrode 310d, a second electrode 310e, a second electrode 310f, a second electrode 310g, and a second electrode 310h. Moreover, in the illustrated example, the detection device 302 may include a second electrode 311a, a second electrode 311b, a second electrode 311c, a second electrode 311d, a second electrode 311e, a second electrode 311f, a second electrode 311g, and a second electrode 311h.

In the illustrated example, the first electrodes 308 may be coupled to the inner side of the first mesh 314a at differing locations and the first electrodes 309 may be coupled to the inner side of the second mesh 314b at differing locations, such as illustrated in FIGS. 3A and 3B. Additionally, in the illustrated example, each of the second electrodes 310 may be coupled to the inner side of the first mesh 314a at a location that is proximate to one of the first electrodes 308 (e.g., as illustrated in FIGS. 3A and 3B) such that each first electrode 308 may correspond with the second electrodes 310. Similarly, in the illustrated example, each of the second electrodes 311 may be coupled to the inner side of the second mesh 314b at a location that is proximate to one of the first electrodes 309 (e.g., as illustrated in FIGS. 3A and 3B) such that each first electrode 309 may correspond to the second electrodes 311. Further, as illustrated in FIG. 3A, the second electrodes 310 and 311 may be electrically coupled to each other such that they each may be associated with a same electrical node of the detection device 302. The electrical coupling between the second electrodes 310 and 311 is not depicted in FIG. 3B merely to aid in readability of FIG. 3B.

For similar reasons as described above with respect to the detection device 102, a number of first electrodes 308 electrically coupled to one or more of the second electrodes 310 may indicate a perspiration rate of the subject. Additionally, a degree of electrical coupling between the first electrodes 308 and the second electrodes 310 may also indicate the perspiration rate of the subject for similar reasons described above with respect to the detection device 302.

Similarly, a number of first electrodes 309 electrically coupled to one or more of the second electrodes 311 may indicate a perspiration rate of the subject. Additionally, a degree of electrical coupling between the first electrodes 309 and the second electrodes 311 may also indicate the perspiration rate.

Further, the electrical coupling of the first electrodes 309 with the second electrodes 311 as compared to the electrical coupling of the first electrodes 308 with the second electrodes 310 may indicate different perspiration rates. For example, perspiration that may reach the first mesh 314a may first pass through the second mesh 314b in some instances. Therefore, perspiration disposed along the inners side of the first mesh 314a may indicate a higher perspiration rate than when perspiration is disposed along the inner side of the second mesh 314b but not the first mesh 314a. As such, electrical coupling of the first electrodes 309 with one or more of the second electrodes 311 may correspond to a higher perspiration rate than electrical coupling of the first electrodes 308 with one or more second electrodes 310.

The control unit 304 may be substantially analogous to the control unit 104 of FIG. 1. Therefore, the control unit 304 may be configured to determine the number of first electrodes 308 and 309 electrically coupled to one or more of the second electrodes 310 and 311, respectively, and may generate a notification signal according to corresponding perspiration rates, similar to as described above with respect to the control unit 104 generating the notification signal. Additionally or alternatively, the control unit 304 may be configured to determine a degree of electrical coupling between one or more of the first electrodes 308 and 309 and one or more of the second electrodes 310 and 311 (e.g., via resistance measurements) and may generate the notification signal according to corresponding perspiration rates. The control unit 304 is not explicitly illustrated in FIG. 3B as being coupled to the electrodes of the detection device 302 as opposed to the depiction in FIG. 3A to aid in readability of FIG. 3B and not to illustrate a different example or embodiment between FIGS. 3A and 3B.

The notification unit 306 may be substantially analogous to the notification unit 106 of FIG. 1. Therefore, the notification unit 306 may be configured to receive the notification signal generated by the control unit 304 and to generate a notification based on the notification signal. The notification may include one or more of the example notifications described above with respect to FIG. 1.

Modifications, additions, or omissions may be made to FIGS. 3A and 3B without departing from the scope of the present disclosure. For example, the system 300 may include any number of other components not expressly illustrated or described. Further, the number of first electrodes and/or second electrodes may vary. Also, in some embodiments, another side of the second mesh 314b that may face the first void and that may be opposite the inner side that may face the second void may include one or more first and second electrodes.

Additionally, the number of mesh layers may vary. Moreover, although the above description is given with respect to the detection device 302 including the first frame 312a and the second frame 312b, in some embodiments, the detection device 302 may include a single frame with the first mesh 314a and the second mesh 314b coupled thereto such that a first void is between the first mesh 314a and the second mesh 314b, while also allowing for a second void between the second mesh 314b and a surface when the corresponding frame is placed against the surface.

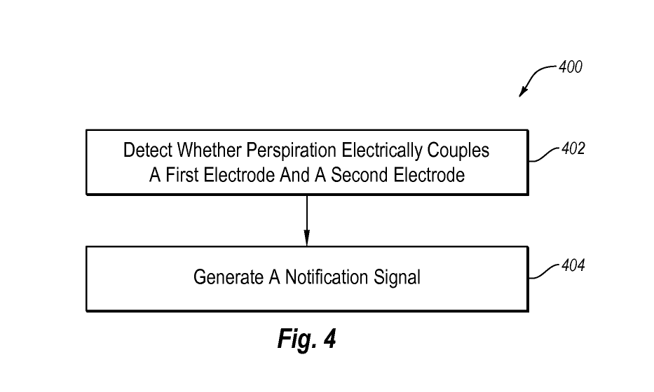

FIG. 4 illustrates a flow diagram of an example method 400 of detecting perspiration and generating a hydration notification, arranged in accordance with at least some embodiments described herein. The method 400 may be performed in whole or in part by one or more of the systems 100, 200, 300, described above, or any other suitable system or apparatus. The method 400 includes various operations, functions, or actions as illustrated by one or more of blocks 402 and/or 404.

For this and other processes and methods disclosed herein, the operations performed in the processes and methods may be implemented in differing order. Furthermore, the depicted operations are only provided as examples, and some of the operations may be optional, combined into fewer operations, supplemented with other operations, or expanded into additional operations without detracting from the essence of the disclosed embodiments. The method 400 may begin at block 402.

In block 402 (“Detect Whether Perspiration Electrically Couples A First Electrode And A Second Electrode”), it may be detected whether perspiration disposed along a mesh electrically couples a first electrode and a second electrode. The first electrode and the second electrode may be coupled to the mesh at different locations of the mesh. Block 402 may be followed by block 404.

In block 404 (“Generate A Notification Signal”), a notification signal may be generated when the perspiration electrically couples the first electrode and the second electrode. In some embodiments, the notification signal may be generated periodically over a time interval in which the perspiration electrically couples the first electrode and the second electrode. Additionally or alternatively, resistance between the first electrode and the second electrode may be measured and the notification signal may be based on the resistance.

Modifications, additions, or omissions may be made to the method 400 without departing from the scope of the present disclosure. For example, in some embodiments, multiple first electrodes may be coupled to the mesh and the method 400 may include generating the notification signal based on a number of the plurality of first electrodes that are electrically coupled to the second electrode. In these or other embodiments, multiple second electrodes may be coupled to the mesh and the method 400 may include generating the notification signal based on a number of the plurality of first electrodes that are electrically coupled to one or more of the plurality of second electrodes.

Additionally, in some embodiments, the method 400 may include generating a notification based on the notification signal. In these or other embodiments, the method 400 may include communicating the notification signal to a notification unit configured to generate the notification. Moreover, in some embodiments, the mesh may be coupled to a frame such that the mesh extends across at least a portion of a space that is outlined by the frame and wherein the method 400 may include placing the frame against a skin surface of a subject.

FIG. 5 is a block diagram illustrating an example computing device 500 that is arranged to direct one or more operations associated with monitoring perspiration and generating corresponding hydration notifications, arranged in accordance with at least some embodiments described herein. The computing device 500 may represent an example configuration of the control units 104, 204, and 304 described above. In a very basic configuration 502, the computing device 500 may include one or more processors 504 and a system memory 506. A memory bus 508 may be used for communicating between the processor 504 and the system memory 506.

Depending on the desired configuration, the processor 504 may be of any type including, but not limited to, a microprocessor (μP), a microcontroller (μC), a digital signal processor (DSP), or any combination thereof. The processor 504 may include one more levels of caching, such as a level one cache 510 and a level two cache 512, a processor core 514, and registers 516. An example processor core 514 may include an arithmetic logic unit (ALU), a floating point unit (FPU), a digital signal processing core (DSP Core), or any combination thereof. An example memory controller 518 may also be used with the processor 504, or in some implementations the memory controller 518 may be an internal part of the processor 504.

Depending on the target configuration, the system memory 506 may be of any type including, but not limited to, volatile memory (such as RAM), non-volatile memory (such as ROM, flash memory, etc.), or any other type of non-transitory computer-readable medium and any combination thereof. The system memory 506 may include an operating system 520, one or more applications 522, and program data 524. The application 522 may include a notification application 526 that may include instructions (executable by a processor such as the processor 504) pertaining to generating the notification signal. The program data 524 may include pressure data 528 that may be useful for determining and obtaining a target pressure and/or other printer-related data, as is described herein. In some embodiments, the application 522 may be arranged to operate with the program data 524 on the operating system 520 such that the pressure adjustment and other printer-related operations may be performed. This described basic configuration 502 is illustrated in FIG. 5 by those components within the inner dashed line.

The computing device 500 may have additional features or functionality, and additional interfaces to facilitate communications between the basic configuration 502 and any required devices and interfaces. For example, a bus/interface controller 530 may be used to facilitate communications between the basic configuration 502 and one or more data storage devices 532 via a storage interface bus 534. Data storage devices 532 may be removable storage devices 536, non-removable storage devices 538, or a combination thereof. Examples of removable storage and non-removable storage devices include magnetic disk devices such as flexible disk drives and hard-disk drives (HDDs), optical disk drives such as compact disk (CD) drives or digital versatile disk (DVDs) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 506, the removable storage devices 536, and the non-removable storage devices 538 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by the computing device 500. Any such computer storage media may be part of the computing device 500.

The computing device 500 may also include an interface bus 540 for facilitating communication from various interface devices (e.g., output devices 542, peripheral interfaces 544, and communication devices 546) to the basic configuration 502 via the bus/interface controller 530. Example output devices 542 include a graphics processing unit 548 and an audio processing unit 550, which may be configured to communicate to various external devices such as a display or speakers via one or more A/V ports 552. Example peripheral interfaces 544 include a serial interface controller 554 or a parallel interface controller 556, which may be configured to communicate with external devices such as input devices (e.g., keyboard, mouse, pen, voice input device, touch input device, etc.) or other peripheral devices (e.g., printer, scanner, etc.) via one or more I/O ports 558. An example communication device 546 includes a network controller 560, which may be arranged to facilitate communications with one or more other computing devices 562 over a network communication link via one or more communication ports 564.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media,” as used herein, may include both storage media and communication media.

The computing device 500 may be implemented as a portion of a small-form factor portable (or mobile) electronic device such as a cell phone, a personal data assistant (PDA), a personal media player device, a wireless web-watch device, a personal headset device, an application-specific device, or a hybrid device that includes any of the above functions. The computing device 500 may also be implemented as a personal computer including both laptop computer and non-laptop computer configurations.

In some examples, a system, may include a mesh that includes a non-electrically conductive mesh material, a first electrode coupled to the mesh at a first location of the mesh, a second electrode coupled to the mesh at a second location of the mesh that is different from the first location; and a control unit electrically coupled to the first electrode and the second electrode and configured to generate a notification signal when the first electrode and the second electrode are electrically coupled.

In some examples, an electrical coupling between the first and second electrodes may be at least in part through the mesh, a substance disposed along a side of the mesh (for example by a liquid substance disposed along a surface of the mesh), or a portion of the mesh modified by a presence of a substance within a vicinity of the mesh (for example, a portion of the mesh having an appreciable electrical conductivity due to the presence of moisture).

In some examples, the mesh may include a hydrophobic material. In some examples, an exterior portion of the mesh may include a hydrophobic surface, which may repel external droplets of water. In some examples, the mesh may include a molded film, a non-woven or woven fabric, or in some examples a material such as a thin film having holes disposed therethrough. In some examples, a mesh may comprise a polymer film with an array (or other plurality) of holes disposed therein. In some examples, a mesh may comprise or be replaced by a porous film, such as a porous polymer film, such as a porous fluoropolymer, for example a porous polytetrafluoroethylene polymer and the like. In some examples, a polymer film, such as a vapor-permeable polymer film, may be used, or other vapor permeable layer. In some examples, a polymer film may be used.

Additionally or alternatively, a silicone based porous film may be used.

In some examples, the system may further include a frame that includes a non-electrically conductive frame material. In some examples, the frame may be configured to support the mesh. In some examples, the frame may be configured to attach or be attached to a surface (e.g., a skin surface of a subject). The mesh may be mechanically coupled to the frame such that the mesh extends across at least a portion of a space defined by the frame. As such the mesh may be spaced apart from the surface such that a void may be formed between the mesh and the surface and enclosed by the frame, the mesh, and the surface. In some examples, a frame may be a generally circular frame with a diameter, such as an interior, average, or exterior diameter (depending on the example) in the range, for example, of 2 mm-20 mm. In some examples, a non-circular frame may have a representative dimension (such as edge length or diameter) in a similar range. Dimensional examples and ranges are not limiting and may be selected, for example, depending on the intended location of the frame on a living subject. In some examples, the frame may support the mesh at a distance spaced apart from, for example, the skin, and this spacing may be, for example, in the range 0.1 mm-10 mm. In some examples, the first electrode and the second electrode may have an electrode spacing, for example, in the range 0.1 mm-20 mm. Example dimensional ranges, such as spacings, are not limiting. In some examples, dimensional ranges may be average dimensions, such as mean spacings, or in some examples may represent the closest spacing. In some examples, the surface may be a vapor-exuding surface, such as a portion of the skin of a living subject that exudes sweat (such as a portion of a human skin, or animal skin such as a dog's paw pad or portion of a horse). The system may be configured so that vapor and/or liquid exuded by the surface may be collected in the void between the surface and the mesh. In some examples, vapor may evaporate from a skin portion of a subject and at least in part condense as liquid on an inner surface of the mesh. In some examples, the condensed liquid may then be collected in a void between the skin portion and the mesh. In some examples, liquid may exude from a skin portion of a subject and at least in part be collected in a void between the skin and the mesh.

As indicated above, the mesh may create a semi-permeable barrier for liquid that may be collected in the void. In some examples, holes in the mesh may be sized such that the surface tension across them may restrict the flow of liquid from inside the void to outside of the void through the mesh without external force (provided by movement) or evaporation. As indicated above, if the void contains sufficient liquid to fill it to an extent that an electrical coupling is made between one or more electrodes (e.g., through the liquid) disposed on an inner side of the mesh that may face the void, a control unit of the system may assume a known volume of liquid in the void. The control unit may compare the known volume to a designed loss rate through the mesh such that a perspiration rate of the subject may be established.

In some examples, the electrodes may be coupled to the mesh through a mechanical contact with a portion of the mesh. In some embodiments, the electrodes may be coupled to an inner side of the mesh that may face the void formed between the mesh and the surface. As described above, electrical coupling may then occur between the electrodes and liquid disposed along the inner side of the mesh when the void is sufficiently full of the liquid. In some embodiments, the mesh may have a metallized or otherwise electrically conducting peripheral portion to facilitate electrical coupling to one or more electrodes.

In some examples, the frame may be a non-electrically conducting material, and may include a polymer, elastomer, and the like. Additionally or alternatively, the frame may include a deformable portion configured to contact the surface, and a relatively more rigid portion configured to support the mesh. In some examples, the frame may include an adhesive layer, allowing the frame to be adhered to the surface.

In some examples, the system may further include a second mesh coupled across the frame at a location different from where the first mesh is coupled across the frame. In some examples, a single frame may support one or more meshes.

In some examples, the mesh may include a polymeric material, as silicones, fluorocarbons (e.g. Teflon), methacrylates, hydrophobic polyesters, a wetting-agent free polycarbonate, any suitable polymer that has been nanotextured to increase a water contact angle, or any other suitable material. The mesh may include holes extending therethrough. The holes may have a hole diameter, for example in the range 0.01 mm-2 mm.

In some examples, the control unit may be further configured to determine whether the first electrode is electrically coupled to the second electrode. Electrical coupling may be determined as an electrical resistance or electrical resistance between first and second electrodes. In some examples, an electrical resistance below a first threshold resistance may indicate electrical coupling. In some examples, an electrical resistance below a second threshold resistance (less than the first threshold resistance) may indicate a problem, such as an electrical short, presence of excess liquid water on the mesh, and the like. As non-limiting examples, the first threshold resistance may be in the range 10 ohms-10 mega-ohms, such as in the range 100 ohms-10 kilo-ohms. The value of the first threshold resistance may depend on the device configuration and also, in some examples, on the subject, and may be adjustable. In some examples, the control unit may be configured to determine a resistance between the first electrode and the second electrode, to generate the notification signal based on the resistance between the first electrode and the second electrode.

In some examples, the control unit may be in communicative coupling (e.g. in wireless communication) with an electronic device. For example, the electronic device may have a screen, on which a person may view data communicated from the control unit. The screen or other transducer of the electronic device may provide an alert to a subject.

In some examples, the system may provide an alert to indicate that a subject should hydrate. For example, the system may include a visual alert (e.g., illumination of a lamp, light emitting diode, and the like), an audible alert (such as an audible alert provided by speaker, piezoelectric buzzer, and the like), a haptic alert (e.g., provided by a vibrating element), or other alert perceptible by a subject, or some combination thereof

The present disclosure is not to be limited in terms of the particular embodiments described herein, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. For example, in the present disclosure electrical resistance is discussed in which a direct current (DC) based system may be implemented. However, in some embodiments, an alternating current based system may be used in which electrical impedance may be substituted for the electrical resistance described. Such modifications and variations are intended to fall within the scope of the appended claims. The present disclosure includes the terms of the appended claims, along with the full scope of equivalents to which such claims are entitled. The present disclosure is not limited to particular methods, reagents, compounds, compositions, or biological systems, which can, of course, vary. The terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Additionally, virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, the disclosure is also thereby described in terms of any individual member or subgroup of members of the Markush group.

For any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. Also all language such as “up to,” “at least,” and the like may include the number recited and refer to ranges which can be subsequently broken down into sub ranges as discussed above. Finally, a range may include each individual member. Thus, for example, a group having 1-3 cells may refer to groups having 1, 2, or 3 cells. Similarly, a group having 1-5 cells refers to groups having 1, 2, 3, 4, or 5 cells, and so forth.

From the foregoing, various embodiments of the present disclosure have been described herein for purposes of illustration, and various modifications may be made without departing from the scope and spirit of the present disclosure. Accordingly, the various embodiments disclosed herein are not intended to be limiting, with the true scope and spirit being indicated by the following claims.

What is claimed is:

1. A system, comprising:
a mesh that comprises a non-electrically conductive mesh material;
a first electrode coupled to the mesh at a first location of the mesh;
a second electrode coupled to the mesh at a second location of the mesh that is different from the first location; and
a control unit electrically coupled to the first electrode and the second electrode and configured to generate a notification signal when the first electrode and the second electrode are electrically coupled to each other.

2. The system of claim 1 further comprising a frame that outlines a space and that includes a non-electrically conductive frame material, wherein the mesh is coupled to the frame such that the mesh extends across at least a portion of the space and such that a void is created between the mesh and a surface when the frame is placed against a surface.

3. The system of claim 2, wherein the surface is a skin surface of a subject.

4. The system of claim 2, wherein the control unit is configured to generate the notification signal when the void fills with a liquid such that the liquid is disposed along the mesh and electrically couples the first electrode and the second electrode.

5. The system of claim 2, wherein the mesh is a first mesh and the device further comprises a second mesh coupled across the frame at a location different from where the first mesh is coupled across the frame such that a void is created within the space between the first mesh and the second mesh.

6. The system of claim 2, wherein the mesh is coupled to the frame such that the mesh extends across and through the space outlined by the frame.

7. The system of claim 2, wherein the frame material comprises silicone.

8. The system of claim 1, wherein the mesh material comprises a hydrophobic material.

9. The system of claim 1, wherein the control unit is further configured to determine whether the first electrode is electrically coupled to the second electrode.

10. The system of claim 1, wherein the control unit is configured to determine an resistance between the first electrode and the second electrode and to generate the notification signal based on the resistance between the first electrode and the second electrode.

11. The system of claim 1, further comprising a plurality of first electrodes that includes the first electrode, wherein each of the plurality of first electrodes is coupled to the mesh.

12. The system of claim 11, wherein the control unit is electrically coupled to each of the plurality of first electrodes and is configured to generate the notification signal based on a number of the plurality of first electrodes that are electrically coupled to the second electrode.

13. The system of claim 11, further comprising a plurality of second electrodes that includes the second electrode, wherein each of the plurality of second electrodes is coupled to the mesh.

14. The system of claim 13, wherein:
the control unit is electrically coupled to each of the plurality of first electrodes;
the control unit is electrically coupled to each of the plurality of second electrodes; and
the control unit is configured to generate the notification signal based on a number of the plurality of first electrodes that are electrically coupled to one or more of the plurality of second electrodes.

15. The system of claim 1, further comprising a notification unit communicatively coupled to the control unit and configured to receive the notification signal and generate a notification based on the notification signal.

16. The system of claim 15, wherein the notification unit includes one or more of a speaker, a vibrator, a light, and a portable electronic device.

17. The system of claim 1, wherein the control unit is configured to periodically generate the notification signal over a time interval in which the liquid substance electrically couples the first electrode and the second electrode.

18. A method comprising:
detecting whether perspiration disposed along a mesh electrically couples a first electrode and a second electrode that are coupled to the mesh at different locations of the mesh; and
generating a notification signal when the perspiration electrically couples the first electrode and the second electrode.

19. The method of claim 18, wherein each of a plurality of first electrodes that includes the first electrode is coupled to the mesh and the method further comprises generating the notification signal based on a number of the plurality of first electrodes that are electrically coupled to the second electrode.

20. The method of claim 18, wherein each of a plurality of first electrodes that includes the first electrode is coupled to the mesh, wherein each of a plurality of second electrodes that includes the second electrode is coupled to the mesh and wherein the method further comprises generating the notification signal based on a number of the plurality of first electrodes that are electrically coupled to one or more of the plurality of second electrodes.

21. The method of claim 18, further comprising periodically generating the notification signal over a time interval in which the perspiration electrically couples the first electrode and the second electrode.

22. The method of claim 18, further comprising generating a notification based on the notification signal.

23. The method of claim 18, further comprising communicating the notification signal to a notification unit configured to generate a notification.

24. The method of claim 23, wherein the notification unit includes one or more of a speaker, a vibrator, a light, and a portable electronic device.

25. The method of claim 18, further comprising determining a resistance between the first electrode and the second electrode and generating the notification signal based on the resistance.

26. The method of claim 18, wherein the mesh is coupled to a frame such that the mesh extends across at least a portion of a space that is outlined by the frame and wherein the method further comprises placing the frame against a skin surface of a subject.
